(19) United States
(12) Patent Application Publication
(10) Pub. No.: US 2008/0305047 A1
(43) Pub. Date: Dec. 11, 2008

(54) Chemosensors Based on Quantum Dots and Oxazine Compounds

(76) Inventors: Francisco M. Raymo, Coral Gable, FL (US); Massimiliano Tomasulo, Miami, FL (US); Ibrahim Yildiz, Miami, FL (US)

Correspondence Address: NIXON & VANDERHYE, PC, 901 NORTH GLEBE ROAD, 11TH FLOOR, ARLINGTON, VA, 22203, US

(21) Appl. No.: 12/158,213

(22) PCT Filed: Dec. 19, 2006
(86) PCT No.: PCT/US2006/048204

§ 371 Date: Aug. 8, 2008

Related U.S. Application Data
(60) Provisional application No. 60/751,311, filed on Dec. 19, 2005.

(51) Int. Cl. A61K 49/00; G01N 21/76; C12Q 1/02

(52) U.S. Cl. 424/9.6; 436/172; 435/29; 977/920

(57) ABSTRACT

We identified a mechanism to detect chemical changes with a modified semiconductor nanoparticle (e.g., an oxazine-adsorbed CdSe—ZnS core-shell quantum dot). Our strategy is based on the chemical transformation of chromo-genie ligands adsorbed on the surface of a quantum dot. This activates an energy transfer pathway from the quantum dot to the adsorbed chromogenic ligands, which causes a change (e.g., increase or decrease) in a characteristic of fluorescent emission (e.g., intensity or lifetime). Thus, modified quantum dots acting through this mechanism can efficiently transduce a chemical event or occurrence into a change in optical signal. Our design can be adapted to signal chemical changes by a diversity of target analytes and, thus, it can be used to develop other fluorescent chemosensors based on the unique properties of quantum dots.

### CROSS REFERENCE TO RELATED APPLICATIONS

This application claims priority benefit of provisional U.S. Application No. 60/751,311, filed Dec. 19, 2005.

### STATEMENT REGARDING FEDERALLY-SPONSORED RESEARCH

The U.S. Government has certain rights in this invention as provided for by the terms of CHE-0237578 awarded by the National Science Foundation.

### BACKGROUND OF THE INVENTION

The invention relates to using an energy transfer pathway from a quantum dot (a signal generator or donor) to an oxazine compound (a sensor/detector or acceptor) in which chemical transformation triggers a change in fluorescence.

The photophysical properties of inorganic quantum dots, which are essentially spherical semiconductor nanocrystals with or without a coating, markedly differ from those of organic chromophore dyes.¹ The formers' molar extinction coefficients, two-photon absorption cross sections, fluorescence lifetimes, and photobleaching resistances are significantly greater than those of dyes. Furthermore, the broad absorption bands of quantum dots extend continuously from the ultraviolet to the visible regions, offering a vast selection of possible excitation wavelengths. In contrast, their narrow emission bands can be precisely positioned within the visible and near-infrared regions by relying on careful adjustments of their diameter. The unique combination of these attractive features continues to stimulate the design of fluorescent probes based on quantum dots for biomedical research.² Indeed, it is becoming apparent that these fluorescent nanoparticles can complement the use of conventional organic fluorophores in a diversity of imaging and sensing applications.

Decades of extensive investigations on the photochemical and photo-physical properties of organic compounds have deduced a number of operating principles to signal target analytes with pronounced fluorescence changes.³ These mechanisms generally transducer the occurrence of a recognition event into a detectable signal by coupling a receptor-substrate association with an electron or energy transfer process. The identification of strategies to extend these protocols from organic fluorophores to quantum dots can eventually lead to the development of fluorescent chemosensors with superior properties. In this context, we have devised a mechanism to transduce the occurrence of a chemical recognition event into a fluorescent signal on the basis of energy transfer from a quantum dot to appropriate ligands adsorbed on their surface. Here, we illustrate our design and demonstrate with a representative example how changes in pH can efficiently switch the fluorescence of our sensitive nanoparticles.

Therefore, it is an objective of the invention to provide improved compositions for sensing a condition or event through a chemical transformation and transducing the occurrence of this condition or event by a fluorescent change. Other advantages and improvements are described below or would be apparent from the disclosure herein.

### SUMMARY OF THE INVENTION

An objective of our invention is a fluorescent chemosensor which can undergo a chemical transformation that results in a change of fluorescence. Further objectives of the invention are described below and in the claims.

A composition of chemosensors is provided. At least one of the chemosensors is comprised of (i) a fluorescent nanoparticle comprising an essentially spherical, semiconductor nanocrystal and (ii) one or more oxazine ligands in optical linkage therewith, such that fluorescence emission from the nanoparticle substantially overlaps with an absorption spectrum of at least one oxazine ligand or its phenolate derivative. An analyte may induce chemical transformation of the oxazine ligand to its phenolate derivative causing a shift in the absorption spectra between the oxazine ligand and the phenolate derivative.

Also provided are processes for using and making these products, which may then be subjected to further processing. It should be noted, however, that any claim directed to a product is not necessarily limited to such processes unless the particular steps of the process are recited in the product claim.

Compounds, compositions, articles (e.g., kits), and processes for using and making the aforementioned products are provided. Other advantages and improvements are described below or would be apparent from the disclosure herein.

### BRIEF DESCRIPTION OF THE DRAWINGS

FIG. 1 illustrates the transformation of [1,3]oxazines 1 and 3 into the hemiaminals 2 and 4, respectively, in the presence of Bu₄NOH.

FIG. 2 shows the absorption spectra of 1 (0.1 mM, MeCN, 20° C.) before (a in FIG. 2) and after (b in FIG. 2) the addition of Bu₄NOH (100 eq.). The emission spectrum (c in FIG. 2) of CdSe—ZnS core-shell quantum dots (0.7 μM, CHCl₃, 20° C., λ_{EX}=375 nm) is also shown.

FIGS. 3A-3B show the absorption spectra of CdSe—ZnS core-shell quantum dots (1.8 μM, CHCl₃, 20° C.) before (a in FIG. 3A) and after (b in FIG. 3A) the attachment of 3 to their surface, and after the consecutive addition of Bu₄NOH (c in FIG. 3A, 4.8 mM) and CF₃CO₂H (d in FIG. 3A, 6.9 mM) to the coated nanoparticles. The emission spectra of CdSe—ZnS core-shell quantum dots (0.9 μM, CHCl₃, 20° C., λ_{EX}=423 nm) coated with 3 are shown before (e in FIG. 3B) and after the consecutive addition of Bu₄NOH (f in FIG. 3B, 2.4 mM) and CF₃CO₂H (g in FIG. 3, 2.3 mM).

FIG. 4 illustrates the transformation of [1,3]oxazines 3a and 5a upon the addition of base or acid.

FIG. 5 shows the absorption spectra of 5a (0.1 mM, MeCN, 20° C.) before (a in FIG. 5) and after the addition of either Bu₄NOH (b in FIG. 5, 100 eq.) or CF₃CO₂H (c in FIG. 5, 16 eq.). Note that 5 in FIG. 5 does not refer to the indole compound used in the synthesis of the model 5a.

FIG. 6 shows the emission intensity of CdSe—ZnS core-shell quantum dots (0.4 μM, 550 μL, CHCl₃, 0.1 M Bu₄NCl, 20° C., λ_{EX}=430 nm, λ_{Em}=552 nm) with (a in FIG. 6) and without (b in FIG. 6) 3 on their surface after the addition of sodium phosphate buffer (200 μL) with pH ranging from 3.2 to 10.7.

### DESCRIPTION OF SPECIFIC EMBODIMENTS OF THE INVENTION

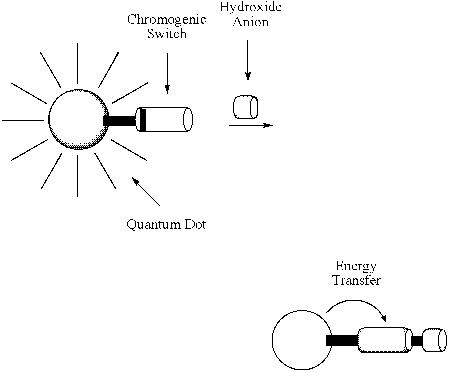

We identify a strategy to switch the fluorescence of a CdSe—ZnS core-shell quantum dot with chromogenic ligands adsorbed on its surface. Each ligand combines a [1,3]oxazine ring and a dithiolane appendage within its molecular skeleton. The dithiolane group anchors the ligand on the ZnS shell of the quantum dot. The [1,3]oxazine ring reacts with a hydroxide anion to generate a 4-nitrophenylazophenolate chromophore. This chromogenic transformation activates an energy transfer pathway from the quantum dot to the adsorbed chromophores. As a result, the fluorescence intensity of the coated quantum dot decreases significantly in the presence of hydroxide anions. In fact, this mechanism can be exploited to probe the pH of aqueous solutions. Indeed, an increase in pH from 7.1 to 8.5 translates into a 35% decrease in the fluorescence intensity of the sensitive quantum dot. Thus, our operating principles for optical switching can efficiently transduce a chemical change into a change (i.e., increase or decrease) in the emissive response of the quantum dot. In principle, this can be extended from hydroxide anions to other target analytes with appropriate adjustments in the molecular design of the chromogenic ligands. It follows that fluorescent chemosensors, based on the unique photophysical properties of quantum dots, can eventually evolve from our design logic and choice of materials.

A chemosensor composition is provided, which is comprised of one or more essentially spherical, fluorescent nanoparticle and a plurality of chromogenic oxazine ligands adsorbed thereon, wherein the fluorescent nanoparticle (energy donor) and one or more of the oxazine ligands or their phenolate derivatives (energy acceptors) are optically linked (i.e., the fluorescent emission from the nanoparticle substantially overlaps with absorption spectra of the ligands or their phenolate derivatives). The presence of an analyte, which is detectable by the chemosensor, induces a chemical transformation (e.g., cleavage) of one or more optically-linked oxazine ligands to their phenolate derivatives. Adsorbed ligands that are inducibly transformed from colorless (i.e., no substantial overlap between the fluorescent emission spectrum and the absorption spectrum of the oxazine compound) to colored (i.e., substantial overlap between the fluorescent emission spectrum and the absorption spectrum of the phenolate derivative) decreases the fluorescent signal, while transformation from colored (i.e., substantial overlap between the fluorescent emission spectrum and the absorption spectrum of the oxazine compound) to colorless (i.e., no substantial overlap between the fluorescent emission spectrum and the absorption spectrum of the phenolate derivative) increases the fluorescent signal. Overlap between spectra is “substantial” when there is a detectable decrease in the fluorescent signal due to absorption by the chromogenic oxazine ligand or its phenolate derivative. A change in fluorescent signal may be detected by measuring intensity (e.g., the number of photons emitted at or around the emission wavelength of the nanoparticle) or lifetime (e.g., the time taken for 30% of the excited states of the nanoparticle to emit and return to ground state). The change may be at least about ±10%, at least about ±20%, at least about ±30%, at least about ±40%, or at least about ±50%.

Nanoparticles are inorganic semiconductor nanocrystals with a mean diameter from about 1 nm to about 10 nm (preferably about 2 nm to about 6 nm) that may or may not be coated. The nanocrystal (i.e., core) is “essentially spherical” as its shape resembles a spheroid (e.g., a porous or solid sphere) described by its diameter that varies by less than about 5% or about 10% as measured across any line through the center of an individual nanocrystal. In a composition, at least 60% of the nanocrystals may deviate less than 5% or 10% in root mean square (rms) diameter from each other. One or more coatings (i.e., shells, each of which are preferably of uniform thickness) may be another inorganic semiconductor and/or an organic layer (e.g., mixed hydrophobic/hydrophilic polymer) that optionally enhances quantum yield of its fluorescence and/or reduces oxidation of the nanocrystal. Exemplary semiconductors are CdS, CdSe, CdTe, ZnSe, ZnTe, and ternary and quaternary mixtures thereof. The other semiconductor coating preferably has a higher band gap energy: e.g., ZnS shell for a CdSe—ZnS core or ZnSe shell for a CdTe—ZnSe core. An exemplary organic layer is SH(CH₂)ₙX, wherein n is from 8 to 15 and X is carboxylate or sulfonate, which aids solubility in aqueous solution. Thus, nanoparticles are preferably water soluble. The wavelength at maximum excitation of a nanoparticle (e.g., from about 300 nm to about 700 nm) may be theoretically calculated from known formulae. The wavelength at maximum emission of a nanoparticle may be in the visible or near infrared of the spectrum (e.g., from about 400 nm to about 900 nm). The difference between wavelengths for maximum absorption by an oxazine ligand and its phenolate derivative may be at least about 150 nm, at least about 175 nm, at least about 200 nm, at least about 225 nm, or at least about 250 nm. The excitation energy may be divided into two photon sources of infrared wavelengths that intersect in a spot of the body, and their energy superimposed.

One or more of the ligands are each comprised of an oxazine compound of the following formula:

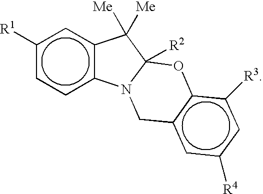

An appendage (R¹) covalently or noncovalently assists in absorbance of the compound on the nanoparticle's surface such that the fluorescent nanoparticle (energy donor) and the chromogenic oxazine ligands or their phenolate derivatives (energy acceptors) are optically linked as described above. Ligands may be covalently adsorbed by amide, ester, or thiol linkage or noncovalently adsorbed by forming a stable metal complex (e.g., a chelate). R² and R³ are examples of functionalities of the ligand that may be varied to bind different target analytes independently or coordinately. Such “receptors” for the chemosensor are chosen to specifically recognize analytes such as, for example, pH ([H⁺]), metal cations (e.g., Na⁺, K⁺, Mg⁺⁺, Ca⁺⁺), glucose, and proteins (e.g., enzymes, hormones). For example, R² and/or R³ may be independently or both hydrogen, alkyl (e.g., methyl, ethyl, butyl, propyl) or cycloalkyl, substituted alkyl or cycloalkyl, aryl (e.g., phenyl), substituted aryl, a heterocycle (e.g., crown ether) or substituted heterocycle, a boronic acid, or a polydentate chelator. R⁴ may be varied to adjust the absorption spectrum of the chromogenic ligand and/or its phenolate derivative. For example, R⁴ may be a chromophore (e.g., nitroso, nitro, azo dyes). On an individual nanoparticle, ligands may be the same or different; the mean number of all ligands adsorbed on an individual nanoparticle may be at least three, at least five, at least seven, or at least nine.

For a given chemical transformation of one or more chromogenic ligands, the diameter of the nanoparticle is chosen to emit a fluorescent signal that substantially overlaps in emission spectrum with the absorption spectra of the one or more ligands in their colored form. The appendage and its mode of attachment to the nanoparticle and the ligand (i.e., covalent or noncovalent) must permit efficient energy transfer between the emitting nanoparticle and the absorbing ligands.

The chemosensor may be added to culture medium, injected into biological fluid (e.g., blood, cerebrospinal fluid, interstitial fluid) or tissue through a catheter or with a syringe, or taken up by a cell or tissue which is then trans-planted into the body. It may be injected directly into a tumor or sequestered from the body's circulatory system into a tumor. Chemosensors and a pharmaceutically-acceptable carrier (e.g., buffered saline solution, water) may be administered (e.g., enteral or parenteral) to a subject (e.g., human or animal) as a medicament or diagnostic agent. It is preferred that compositions for in vivo or long-term in vitro use be apyrogenic (e.g., less than about 0.25 endotoxin units per mL) and aseptic (e.g., less than one cultured infectious microbe per mL).

A kit may be provided comprised of one or more containers in a package with (i) one or more chemosensors in container(s) and optionally one or more of (ii) a physiologically-acceptable carrier or other solvent and (iii) a calibration standard of a known amount(s) of analyte in a container(s). A composition may be in a container(s) or may be made from components therein. Other optional components of the kit includes (iv) a transfer pipet, (v) a reaction vessel (e.g., transparent multiwell plate, vial), (vi) a means for sampling, and (vii) written instructions for detecting analyte.

A method of detecting at least the presence or quantity of an analyte is provided comprising (a) contacting at least one chemosensor with a solution which might contain an analyte, wherein the analyte induces a chemical trans-formation of the oxazine ligand to its phenolate derivative; (b) measuring whether or not there is a detectable change in fluorescent signal of the chemosensor; and (c) correlating the change in the fluorescent signal with detection of the analyte.

For a fixed amount or concentration of analyte, fluorescence may be measured at one or more wavelengths. A blank sample containing a diluent (for a liquid sample) or an eluent (for a solid sample) may serve as a negative control (e.g., to subtract background from test samples). Known quantities of analyte may serve as positive controls (e.g., a standard for confirming sensitivity to the presence of analyte or calibrating the quantitation of an amount or concentration of analyte in a sample). Varying the quantity (e.g., amount or concentration) of a chemosensor may have a different range of analyte quantities that can be determined.

In an assay, fluorescence may be measured and correlated with the presence of analyte in a sample. Sensitivity of the assay for analyte may be measured by performing a dilution series of a known quantity of analyte and determining the minimal amount or concentration that will be reliably detected under test conditions. A liquid suspected of containing analyte may be put into a diluent, or concentrated by evaporation or reverse osmosis prior to assay. The chemical reaction is then performed by mixing the liquid sample with the other components of the reaction. A solid that is suspected of containing analyte may be “sampled” by soaking the solid in eluent to extract at least some analyte that might be present.

Nanoparticles may be selectively activated by spatial- and/or temporal-specific excitation (see below); oxazine ligands may be selectively activated by the analyte. Fluorescence emission may be visualized using a fluorescent microscope or another detector (e.g., camera) with the fluorescent signal recorded on a CCD diode array or photographic emulsion. Magnification and recording provide spatial and temporal resolution, respectively, of individual nanoparticles. Chemosensors may also be examined for their optical characteristics by spectroscopy using a diode detector or photomultiplier tube. Nanoparticles may be excited by a light source (e.g., dye or gas laser, lamp, light emitting diode) focused on an illuminated portion of the microscope field or body. Fluorescent signal may be separated for analysis with bandpass filters and/or dichroic glass. Moreover, events may be chronologically resolved by exciting the nanoparticle at a specific time for limited duration.

In vitro cell cultures may be visualized. For in vivo imaging, optical fiber may be introduced into a subject (e.g., human or animal) and the local area illuminated, or the body or part thereof may be illuminated with a light box. A CdTe nanocrystal and two-photon excitation are preferred for noninvasive imaging in the near infrared of the spectrum. Two or three light beams may be focused in a spot (perhaps at a specific time for limited duration) to locally excite nanoparticles. Individual images may be directly examined or they may be digitally processed (e.g., tomography).

Molecules (e.g., membrane lipids, nucleic acids, proteins) or intact cells may be fluorescently tagged using the chemosensors (e.g., the molecule can be attached as R²). Their movement may be visualized in culture or the body, and chemical changes may be detected. Cell proliferation or movement may be monitored in cancer and metastasis, inflammation, or functioning of the lympho-hematopoietic system. Transport and recycling of molecules within the cell or trafficking of cells within the body may be visualized, and chemical changes may be detected. Receptor-ligand (e.g., protein growth factors or hormones, steroids) interaction may be monitored. Cells may be tagged on their membranes or loaded internally, and then traced as they circulate or differentiate in the body. Stem cells may be tracked and asymmetric cell division may be studied.

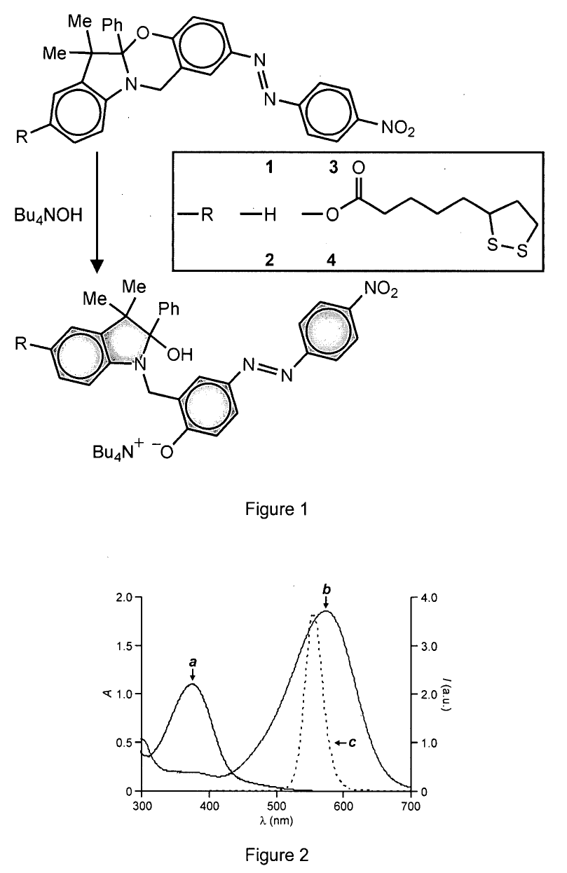

We discovered that the [1,3]oxazine 1 compound (FIG. 1) reacts with Bu₄NOH to form the hemiaminal 2 compound.⁴ This quantitative transformation causes pronounced changes in the ultraviolet and visible regions of the absorption spectrum. Specifically, the spectrum of 1 (a in FIG. 2) shows a band at 375 nm, which disappears after treatment with Bu₄NOH (b in FIG. 2). Concomitantly, an intense band for the 4-nitrophenylazophenolate chromophore of 2 appears at 574 nm. The drastic increase in absorption wavelength with the transformation of 1 into 2 can, in principle, be exploited to activate an energy transfer process. For example, a fluorescent partner with an emission band centered between 550 and 600 nm can donate excitation energy to 2, but not to 1. This design requirement happens to be satisfied by CdSe—ZnS core-shell quantum dots with a mean diameter of about 2.9 nm.⁵ Indeed, their narrow emission at 555 nm (c in FIG. 2) overlaps the absorption of 2 (b in FIG. 2), but not that of 1 (a in FIG. 2). Thus, the excitation energy of the quantum dot can be efficiently transferred to 2, if donor and acceptor are constrained in close proximity.

The affinity of the two sulfur atoms of lipoic acid derivatives for metals can be exploited to adsorb organic molecules on gold nanoparticles⁶ and CdSe—ZnS core-shell quantum dots.⁷ Based on these considerations, we designed the [1,3]oxazine compound 3 (FIG. 1) and synthesized this compound in five steps starting from commercially-available precursors. The treatment of a pre-formed CdSe—ZnS core-shell quantum dot with 3 in CHCl₃ encourages the noncovalent attachment of this organic ligand on the surface of the semiconductor nanoparticle.

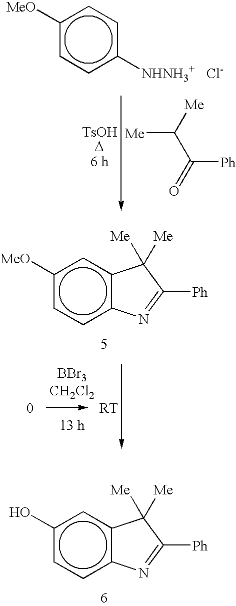

Chemicals were purchased from commercial sources and were used as received with the exception of MeCN and CH₂Cl₂, which were distilled over CaH₂. All reactions were monitored by thin-layer chromatography, using aluminum sheets coated with silica (60, F₂₅₄). The fast atom bombardment mass spectra (FABMS) were recorded with a VG Mass Lab Trio-2 in a 3-nitrobenzyl alcohol matrix. The nuclear magnetic resonance (NMR) spectra were recorded with a Bruker Avance 300, 400 or 500 spectrometer. The absorption spectra were recorded with a Varian Cary 100 Bio spectrometer using quartz cells with a path length of 0.5 cm. Emission spectra were recorded with a Varian Cary Eclipse spectrometer in aerated solutions. Luminescence lifetimes were measured with an Edinburgh Instruments FL920 spectrometer in aerated solutions.

2-Phenyl-3,3′-dimethyl-5-methoxy-3H-indole (5). A solution of i-propylphenyl-ketone (2.2 mL, 14 mmol), 4-methoxyphenylhydrazine hydrochloride (2.5 g, 14 mmol), and 4-toluenesulfonic acid (27 mg, 0.1 mmol) in EtOH (30 mL) was heated under reflux for 6 hr. After cooling to ambient temperature, aqueous NaHCO₃ (5 wt %, 10 mL) was added and the mixture was extracted with CH₂Cl₂ (4×10 mL). Then, the solvent was distilled off under reduced pressure and the residue was purified by column chromatography [SiO₂:hexane/MeCO₂Et 1:1 (v/v)] to provide 5 (2.15 g, 60%) as a brown-yellow oil. FABMS: m/z=251 [M+H]⁺; ¹H-NMR (300 MHz, CDCl₃): δ=1.59 (6H, s), 3.82 (3H, s), 6.89-6.91 (2H, m), 7.47-7.49 (3H, m), 7.64 (1H, d, 9 Hz), 8.10-8.13 (2H, m); ¹³C-NMR (75 MHz, CDCl₃): δ=25.1, 53.3, 55.9, 107.7, 112.6, 121.5, 128.2, 128.5, 128.8, 130.4, 133.6, 149.5, 158.8, 181.5.
2-Phenyl-3,3′-dimethyl-5-hydroxy-3H-indole (6). A solution of BBr₃ (1 M) in CH₂Cl₂ (6 mL) was added dropwise to a solution of 5 (0.9 g, 4 mmol) in CH₂Cl₂ (30 mL) maintained at 0° C. under Ar. After 1 hr, the mixture was allowed to warm to ambient temperature and was maintained under these conditions for a further 12 hr. Then, a saturated aqueous solution of NaHCO₃ (15 mL) was added and the resulting mixture was extracted with CH₂Cl₂ (3×20 mL). The solvent was distilled off under reduced pressure to provide 6 (0.75 g, 83%) as a green solid. FABMS: m/z=238 [M+H]⁺; ¹H-NMR (400 MHz, CDCl₃): δ=1.64 (6H, s), 6.90 (1H, dd, 8 and 2 Hz), 6.97 (1H, d, 2 Hz), 7.55-7.58 (3H, m), 7.71 (1H, d, 8 Hz), 8.24 (2H, d, 8 Hz); ¹³C-NMR (100 MHz, CDCl₃): δ=25.9, 31.8, 53.8, 109.5, 115.7, 120.8, 128.4, 129.2, 129.5, 130.3, 132.8, 148.3, 156.9, 181.7.

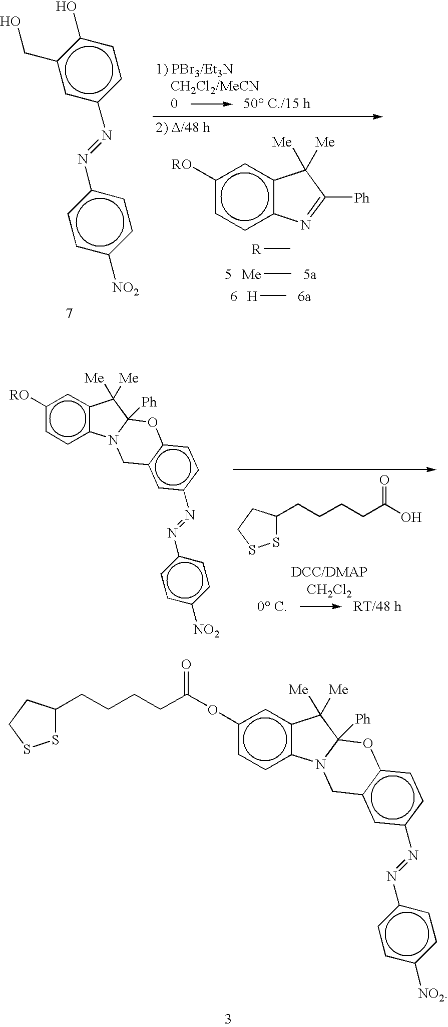

2-(4′-Nitrophenylazo)-5a-phenyl-6,6-dimethyl-5a,6-dihydro-8-methoxy-12H-indolo[2,1-b][1,3]benzooxazine (5a). A solution of PBr₃ (26 μL, 0.3 mmol) in CH₂Cl₂ (300 μL) was added dropwise to a solution of 7 (100 mg, 0.4 mmol) in MeCN (20 mL) maintained at 0° C. under Ar. After the addition of Et₃N (77 μL, 0.6 mmol), the mixture was allowed to warm to ambient temperature over 3 h and then it was heated at 50° C. for 12 h. After the subsequent addition of 5 (112 mg, 0.5 mmol), the mixture was heated under reflux for 48 h. After cooling to ambient temperature, the solvent was distilled off under reduced pressure and the residue was purified by column chromatography [SiO₂:CH₂Cl₂/hexane 3:1 (v/v)] to provide 5a (47 mg, 25%) as an orange solid. FABMS: m/z=507 [M+H]⁺; ¹H-NMR (500 MHz, CDCl₃): δ=0.86 (3H, s), 1.59 (3H, s), 3.78 (3H, s), 4.55 (1H, d, 17 Hz), 4.60 (1H, d, 17 Hz), 6.64-6.70 (2H, m), 6.80 (1H, d, 2 Hz), 6.97 (1H, d, 9 Hz), 7.34-7.42 (3H, m), 7.64 (1H, d, 2 Hz), 7.38-7.73 (3H, m), 7.91 (2H, d, 9 Hz), 8.33 (2H, d, 9 Hz); ¹³C-NMR (75 MHz, CDCl₃): δ=18.8, 27.9, 41.5, 50.2, 56.2, 105.4, 109.6, 110.4, 111.8, 118.8, 120.7, 122.6, 123.4, 124.5, 125.1, 128.6, 128.8, 129.1, 136.8, 139.8, 141.4, 146.7, 148.6, 155.0, 156.4, 158.1.
2-(4′-Nitrophenylazo)-5a-phenyl-6,6-dimethyl-5a,6-dihydro-8-hydroxy-12H-indolo[2,1-b][1,3]benzooxazine (6a). A solution of PBr₃ (18 μL, 0.2 mmol) in CH₂Cl₂ (160 μL) was added dropwise to a solution of 7 (60 mg, 0.2 mmol) in MeCN (15 mL) maintained at 0° C. under Ar. After the addition of Et₃N (50 μL, 0.4 mmol), the mixture was allowed to warm to ambient temperature over 3 hr and then it was heated at 50° C. for 12 hr. After the subsequent addition of 6 (71 mg, 0.3 mmol), the mixture was heated under reflux for 48 hr. After cooling to ambient temperature, the solvent was distilled off under reduced pressure and the residue was purified by column chromatography [SiO₂:CH₂Cl₂/hexane 3:1→5:1 (v/v)] to provide 6a (27 mg, 25%) as an orange solid. FABMS: m/z=493 [M+H]⁺; ¹H-NMR (400 MHz, CDCl₃): δ=0.85 (3H, s), 1.57 (3H, s), 4.43 (1H, s), 4.53 (1H, d, 16 Hz), 4.58 (1H, d, 16 Hz), 6.57-6.63 (2H, m), 6.72 (1H, d, 2 Hz), 6.95 (1H, d, 9 Hz), 7.32-7.43 (3H, m), 7.63 (1H, d, 2 Hz), 7.67-7.69 (2H, m), 7.73 (1H, dd, 2 and 9 Hz), 7.91 (2H, d, 9 Hz), 8.33 (2H, d, 9 Hz); ¹³C-NMR (100 MHz, CDCl₃): δ=18.7, 27.8, 41.5, 50.2, 105.4, 109.8, 111.2, 113.9, 118.8, 120.7, 122.6, 123.4, 124.5, 125.1, 128.5, 128.8, 129.1, 136.7, 140.0, 141.4, 146.7, 148.6, 150.4, 156.4, 158.1.
2-(4′-Nitrophenylazo)-5a-phenyl-6,6-dimethyl-5a,6-dihydro-8-(1′,2′-dithiolane-3′)-pentacarboxy-12H-indolo[2,1-b][1,3]-benzooxazine (3). A solution of N,N′-dicyclohexylcarbodiimide (DCC, 11 mg, 0.05 mmol) was added dropwise to a solution of 6a (23 mg, 0.05 mmol), (±)-α-lipoic acid (11 mg, 0.05 mmol), and 4-dimethylaminopyridine (DMAP, 1 mg) in CH₂Cl₂ (25 mL) maintained at 0° C. under Ar. After 1 hr, the mixture was allowed to warm to ambient temperature and maintained under these conditions for a further 48 hr. Then, the solvent was distilled off under reduced pressure and the residue was purified by column chromatography [SiO₂:CH₂Cl₂/hexane 4:1→6:1 (v/v)] to provide 3 (32 mg, 94%) as an orange solid. FABMS: m/z=0.681 [M+H]⁺; ¹H-NMR (400 MHz, CDCl₃): δ=0.86 (3H, s), 0.90-0.92 (2H, m), 1.58 (3H, s), 1.75-1.80 (4H, m), 1.93-1.95 (1H, m), 2.46-2.50 (1H, m), 2.56 (2H, t, 7 Hz), 3.12-3.20 (2H, m), 3.59-3.63 (1H, m), 4.57 (1H, d, 17 Hz), 4.63 (1H, d, 17 Hz), 6.69 (1H, d, 9 Hz), 6.85 (1H, dd, 2 and 8 Hz), 6.90 (1H, d, 2 Hz), 6.98 (1H, d, 9 Hz), 7.34-7.43 (3H, m), 7.63 (1H, d, 2 Hz), 7.65-7.67 (2H, m), 7.74 (1H, dd, 2 and 9 Hz), 7.92 (2H, d, 9 Hz), 8.34 (2H, d, 9 Hz); ¹³C-NMR (100 MHz, CDCl₃): δ=14.5, 18.8, 25.1, 27.9, 29.1, 34.5, 35.1, 38.9, 40.6, 50.1, 56.7, 105.2, 109.4, 116.6, 118.9, 120.4, 120.5, 122.5, 123.4, 124.6, 125.1, 128.5, 128.9, 129.2, 136.5, 139.4, 145.0, 145.3, 146.8, 148.6, 156.4, 157.9, 172.8.
CdSe—ZnS Core-Shell Quantum Dots. A mixture of CdO (51 mg, 0.4 mmol), n-tetradecylphosphonic acid (223 mg, 0.8 mmol), and tri-n-octylphosphine oxide (3.78 g, 9.8 mmol) was heated at 320° C. under Ar until a clear solution was obtained. Then, the temperature was lowered to 220° C. and a solution of Se (41 mg, 0.5 mmol) in tri-n-octylphosphine (2.4 mL) was added to initiate crystallization of nanoparticles. After the addition, the mixture was maintained at 200° C. for 40 min with periodic monitoring of the time-dependent growth of the nanoparticles. A narrow range of diameters can be grown: decreasing the time of synthesis will decrease their mean diameter while increasing the time of synthesis will increase their mean diameter. The temperature was then lowered to 120° C. to stop crystal growth and a solution of ZnEt₂ (1.6 mL, 0.16 mmol) and hexamethyldisilathiane (0.30 mL, 1.4 mmol) in tri-n-octylphosphine (5 mL) was added dropwise. After the addition, the mixture was maintained at 70° C. for 5 hr. After cooling to ambient temperature, MeOH (200 mL) was added and the resulting precipitate was filtered and dissolved in CHCl₃ (50 mL). This procedure was repeated three more times and then the solvent was distilled off under reduced pressure to provide CdSe—ZnS core-shell quantum dots (367 mg) as a reddish powder.
Modified CdSe—ZnS Core-Shell Quantum Dots. A solution of CdSe—ZnS core-shell quantum dots (12 mg) and 3 (35 mg) in CHCl₃ (20 mL) was heated under reflux for 24 hr. After cooling to ambient temperature, the solvent was distilled off under reduced pressure. The residue was suspended in MeCN (8 mL) and the supernatant was removed after centrifugation. This procedure was repeated three more times to provide modified CdSe—ZnS core-shell quantum dots (10 mg) as a reddish powder.

The absorption spectrum of the modified CdSe—ZnS core-shell quantum dots shows the band-gap absorption to be centered at 540 nm. This value corresponds to a diameter of about 2.9 nm for the CdSe core. The emission spectrum reveals a narrow and symmetric band centered at 552 nm with a quantum yield (Φ) of 0.28. The luminescence intensity decays biexponentially on a nanosecond time scale. The lifetimes (τ₁ and τ₂) are 23.1 ns and 9.9 ns with fractional contributions (ƒ₁ and ƒ₂) of 0.54 and 0.46, respectively. These values correspond to an average lifetime ( τ̅) of 17.1 ns.

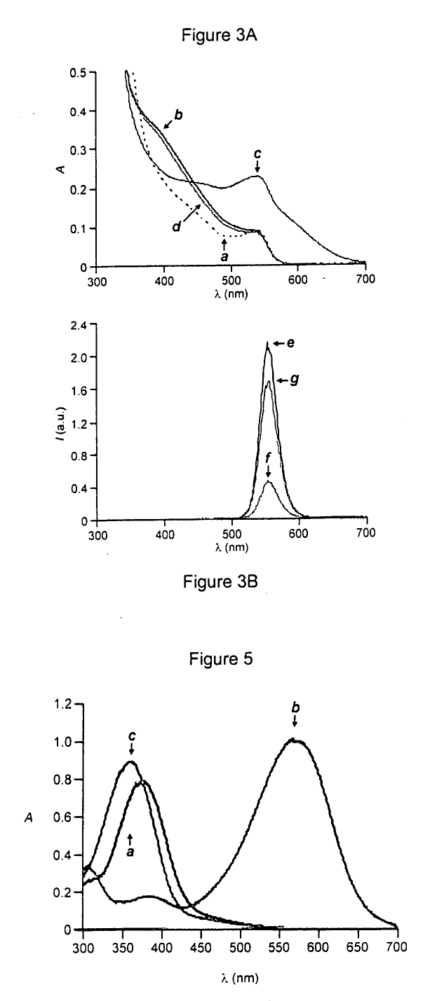

Consistently, the absorption spectra, recorded before (a in FIG. 3) and after (b in FIG. 3) exposing the quantum dots to 3, show the appearance of a band at 392 nm. This band resembles that of 1 (a in FIG. 2) and its absorbance, relative to that of the quantum dot at 542 nm, indicates that each semiconductor nanoparticle is coated by an average of 6 to 7 [1,3]oxazine molecules.⁸

The addition of Bu₄NOH to a solution of modified quantum dots encourages the transformation of 3 into 4 (FIG. 1) on the ligand-covered surface of the quantum dot. Concomitantly, the absorption spectrum (c in FIG. 3) reveals the appearance of the characteristic band at 563 nm for the 4-nitrophenylazo-phenolate chromophore of 4. This band resembles that of 2 (b in FIG. 2) and overlaps the emission of the quantum dot (c in FIG. 2). In fact, the pronounced absorbance increase in this range of wavelengths activates an energy transfer pathway from the quantum dot to the surrounding 4-nitrophenylazophenolate acceptors. As a result, the fluorescence of the modified quantum dot decreases by about 55% (d and e in FIG. 3) in the presence of Bu₄NOH.

The absorption spectra of the quantum dots, recorded before and after the adsorption of 3 through its dithiolane anchoring group, show an increase in absorbance at 398 nm, where the chromophore of 3 absorbs. On the basis of this absorbance increase at 398 nm relative to the band-gap absorbance of the quantum dots, the average number of ligands per nanoparticle can be estimated to be about 6 to 7. The emission spectra of the quantum dots, recorded before and after adsorption of 3, reveal a significant decrease in luminescence intensity at 552 nm. In fact, Φ drops from 0.28 to 0.12 and τ̅ decreases from 17.1 ns to 10.4 ns with the attachment of the ligand. This partial luminescence quenching is consistent with the expected photoinduced electron transfer from the indole fragment of the organic ligand to the excited inorganic nanoparticle.

Chromogenic transformation of organic ligands on the surface of an inorganic quantum dot efficiently transduces a chemical transformation induced by hydroxide anions (3a into 3b) into a detectable change in fluorescence intensity. The very same process can be exploited to probe the pH of aqueous solutions in two-phase systems. Specifically, aliquots of sodium phosphate buffer at fixed pH can be added to a CHCl₃ solution of the modified quantum dots. After vigorous mixing, the quantum dots in the organic phase adjust their fluorescence intensity to the pH of the aqueous phase. In fact, the intensity at 555 nm decreases by about 35% with an increase in pH from 7.1 to 8.5. The fading in fluorescence with the gradual increase in pH is also observed visually under ultraviolet illumination. In contrast, quantum dots lacking the chromogenic ligand 3 on their surface are insensitive to the pH of the aqueous phase under otherwise identical conditions. Thus, the chromogenic and pH-sensitive organic ligands are responsible for the regulation of the emissive behavior of the inorganic nanoparticle. At pH values below 7, chromogenic transformation was also efficiently transduced.

Following on our initial studies that adsorption of [1,3]oxazine ligands on the surface of a quantum dot results in partial luminescence quenching, those studies were expanded. Electron transfer from the indole fragment to the nanoparticle is mainly responsible for the decrease in luminescence intensity. Upon addition of base, the [1,3]oxazine ring of the ligands opens to generate a 4-nitrophenylazophenolate chromophore, which absorbs in the range of wavelengths where the quantum dot emits. This transformation activates an energy transfer pathway from the excited nanoparticle to the ligands. In addition, the oxidation potential of the ligand shifts in the negative direction improving the efficiency of electron transfer. The overall result is a decrease in the luminescence quantum yield of 83%. The addition of acid also opens the [1,3]oxazine ring of the ligands. But the resulting 4-nitrophenylazophenol does not absorb in the visible region and cannot accept energy from the excited nanoparticles. Furthermore, the oxidation potential shifts in the positive direction, lowering the electron transfer efficiency. In fact, the luminescence quantum yield increases by about 33% as a result of this transformation. These changes are fully reversible and can be exploited to probe the pH of aqueous solutions from about 3 to about 11. Indeed, our sensitive chemosensors adjust their luminescence in response to variations in pH within this particular range of values.

Our heterocyclic compounds incorporate a [1,3]oxazine ring at their core. Upon addition of Bu₄NOH base, the [1,3]oxazine ring opens to generate a phenolate chromophore. This process is accompanied by the appearance of an intense band in the visible region of the absorption spectrum. In principle, this chromogenic transformation can be exploited to regulate the luminescence of a complementary quantum dot on the basis of energy transfer. Indeed, the elemental composition and diameter of these inorganic nanoparticles can be adjusted to ensure an optimal overlap between their emission band and the developing absorption of our chromogenic [1,3]oxazines. If the inorganic and organic components are then constrained in close proximity, the chromogenic transformation of the latter can be exploited to switch off the luminescence of the former. Thus, the resulting system should be able to sense the absence or presence of a target analyte with significant changes in luminescence intensity.

The affinity of the dithiolane ring for CdSe—ZnS core-shell quantum dots can be exploited to encourage the adsorption of organic compounds on the surface of these nanoparticles. An aliphatic tail terminated by a dithiolane ring was appended to our chromogenic [1,3]oxazine relying on an ester linkage. In particular, we designed the target ligand 3a and its model 5a, and prepared these compounds starting from the 4-nitrophenylazophenol 7 and the indoles 5 and 6. Specifically, the bromination of 7 with PBr₃ in the presence of Et₃N and the reaction of the resulting bromide with either 5 or 6 in situ gives [1,3]oxazine 5a or 6a, respectively. Esterification of 6a with (±)-α-lipoic acid in the presence of N,N′-dicyclohexylcarbodiimide (DCC) and 4-dimethylaminopyridine (DMAP) then yields the target ligand 3a.

Diverse electron acceptors and donors have been observed to exchange electrons with the core of core-shell quantum dots upon excitation despite the presence of an insulating shell around it. In particular, tertiary amines can transfer electrons to the emissive core of CdSe—ZnS nanoparticles upon excitation with concomitant luminescence quenching. Thus, the relatively electron-rich nitrogen atom of the indole fragment of 3a can in principle donate electrons to the core of these nanoparticles. Consistently, the cyclic voltammogram of the model 5a shows an oxidation wave at +0.88 V vs. Ag/AgCl. This wave can be assigned to oxidation of the indole fragment of 5a. Upon addition of Bu₄NOH, 5a is converted into 5c and the oxidation wave moves to +0.48 V. In contrast, the transformation of 5a into 5c shifts the oxidation potential in the opposite direction. Indeed, the oxidation wave moves to +1.35 V after the addition of CF₃CO₂H. Thus, these changes in oxidation potential suggest that the ability of the organic ligand 3a to transfer electrons to CdSe—ZnS core-shell quantum dots upon excitation should increase after the addition of base and decrease after the addition of acid.

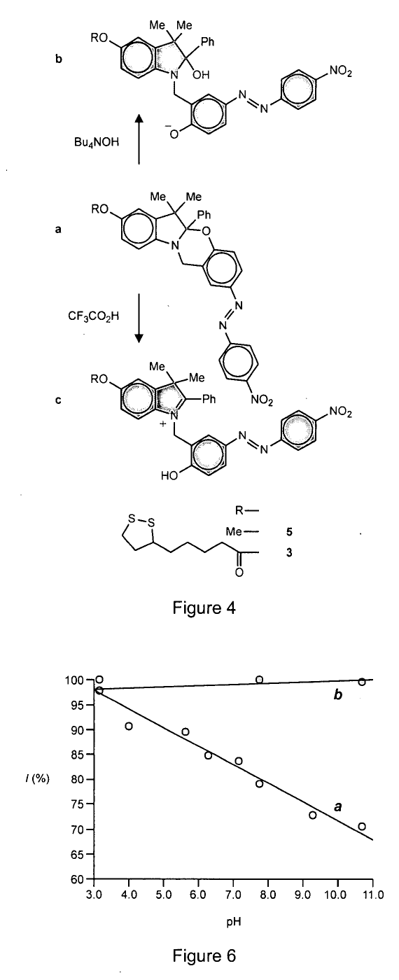

The addition of Bu₄NOH to the coated CdSe—ZnS core-shell quantum dots induced the opening of the [1,3]oxazine ring of 3a with the formation of 3b (see FIG. 4). The 4-nitrophenylazophenolate chromophore of 3b absorbs in the visible region. As a result, a new band appears at 553 nm in the absorption spectrum of the quantum dots. This absorption overlaps the emission of the quantum dots and, therefore, the transformation of 3a into 3b on the surface of the nanoparticles activates an energy transfer pathway from the inorganic to the organic components. The emission intensity at 552 nm decreases significantly, Φ drops from 0.12 to 0.02 and τ̅ changes from 10.4 ns to 9.3 ns. It is important to stress, however, that the transformation of 3a into 3b shifts the oxidation potential in the negative direction by 0.40 V. This change facilitates the photoinduced electron transfer from the organic ligands to the inorganic nanoparticle. Thus, the luminescence quenching increases with the conversion of 3a into 3b because of the activation of an energy transfer pathway and also for the enhancement in the electron transfer efficiency.

The addition of CF₃CO₂H to the coated CdSe—ZnS core-shell quantum dots induced the opening of the [1,3]oxazine ring of 3a with the formation of 3c (see FIG. 4). The 4-nitrophenylazophenol chromophore of 3c absorbs mainly at ultraviolet wavelengths. As a result, the absorption spectra of the quantum dots do not show any significant change in the visible region with the transformation of 3a into 3c. Thus, the excitation energy of the nanoparticles cannot be transferred to the adsorbed ligands neither before nor after the conversion of 3a into 3c. In addition, this process shifts the oxidation potential of the ligand in the positive direction by 0.47 V, inhibiting the photoinduced electron transfer from the organic ligand 3a to transfer electrons to CdSe—ZnS core-shell quantum dots upon excitation should increase after the addition of base and decrease after the addition of acid.

The addition of Bu₄NOH to the coated CdSe—ZnS core-shell quantum dots induced the opening of the [1,3]oxazine ring of 3a with the formation of 3b (see FIG. 4). The 4-nitrophenylazophenolate chromophore of 3b absorbs in the visible region. As a result, a new band appears at 553 nm in the absorption spectrum of the quantum dots. This absorption overlaps the emission of the quantum dots and, therefore, the transformation of 3a into 3b on the surface of the nanoparticles activates an energy transfer pathway from the inorganic to the organic components. The emission intensity at 552 nm decreases significantly, Φ drops from 0.12 to 0.02 and τ̅ changes from 10.4 ns to 9.3 ns. It is important to stress, however, that the transformation of 3a into 3b shifts the oxidation potential in the negative direction by 0.40 V. This change facilitates the photoinduced electron transfer from the organic ligands to the inorganic nanoparticle. Thus, the luminescence quenching increases with the conversion of 3a into 3b because of the activation of an energy transfer pathway and also for the enhancement in the electron transfer efficiency.

The addition of CF₃CO₂H to the coated CdSe—ZnS core-shell quantum dots induced the opening of the [1,3]oxazine ring of 3a with the formation of 3c (see FIG. 4). The 4-nitrophenylazophenol chromophore of 3c absorbs mainly at ultraviolet wavelengths. As a result, the absorption spectra of the quantum dots do not show any significant change in the visible region with the transformation of 3a into 3c. Thus, the excitation energy of the nanoparticles cannot be transferred to the adsorbed ligands neither before nor after the conversion of 3a into 3c. In addition, this process shifts the oxidation potential of the ligand in the positive direction by 0.47 V, inhibiting the photoinduced electron transfer from the organic ligand to the inorganic nanoparticle. In fact, the emission intensity at 552 nm increases after the formation of 3c with an enhancement in 4 from 0.12 to 0.18 and in τ̅ from 10.4 ns to 12.2 ns.

The emission behavior of a CdSe—ZnS core-shell quantum dot conjugated to 3a demonstrates that the transformations of the organic ligand in the presence of either acid or base regulate the luminescence intensity of the inorganic nanoparticles. In fact, these sensitive nanostructures can be employed to probe the pH of aqueous solutions in biphasic systems with the assistance of a phase transfer catalyst. Specifically, the luminescence intensity of a CHCl₃ phase containing Bu₄NCl and the coated quantum dots varies with the pH of an overlaid aqueous phase (a in FIG. 6). Under these conditions, a pH increase from 3.2 to 10.7 in the aqueous phase translates into a luminescence decrease of about 29% in the organic phase. Indeed, the ligand 3c is converted into 3a and then eventually into 3b as the pH increases with a concomitant decrease in Φ. Instead, the luminescence remains essentially unaffected over the same pH range (b in FIG. 6) when the CdSe—ZnS core-shell quantum dots are not coated with 3a. Thus, the pH dependence of the luminescence intensity is, indeed, a result of the presence of the organic ligands on the surface of the inorganic nanoparticles.

The adsorption of pH-sensitive [1,3]oxazines on the surface of CdSe—ZnS core-shell quantum dots offers the opportunity to switch the luminescence of these nanoparticles by chemical stimulation. Indeed, the [1,3]oxazine rings of the organic ligands open upon addition of base to generate 4-nitrophenylazo-phenolate chromophores. This transformation activates an energy transfer pathway from the excited quantum dot to the ligands, and facilitates electron transfer in the opposite direction. As a result, the luminescence quantum yield decreases by 85%. The addition of acid also opens the [1,3]oxazine ring. But the resulting 4-nitrophenylazophenol groups cannot accept the excitation energy of the nanoparticles and are poor electron donors. Consistently, the luminescence quantum yield increases by 33%. Thus, the organic ligands adsorbed on the inorganic nanoparticles impose pH sensitivity on their emissive behavior. In fact, these quantum dots can probe the pH of aqueous solutions by adjusting their luminescence intensity to pH changes in the 3-11 range. Similar organic ligands can be designed to alter their absorption and redox properties in response to target analytes other than hydroxide anions and protons, regulating the luminescence of the associated quantum dots as a result. Therefore, a new generation of luminescent chemosensors based on the unique photophysical properties of quantum dots can ultimately emerge on the basis of these operating principles.

### REFERENCES

1. (a) Bawendi, M. G.; Steigerwald, M. L.; Brus, L. E. Ann. Rev. Phys. Chem. 1990, 41, 477-496. (b) Alivisatos, A. P. Science 1996, 271, 933-937. (c) Efros, A. L.; Rosen, M. Ann. Rev. Mater. Sci. 2000, 30, 475-521. (d) Yoffe, A. D. Adv. Phys. 2001, 50, 1-208. (e) Burda, C.; Chen, X. B.; Narayana, R.; El-Sayed, M. A. Chem. Rev. 2005, 105, 1025-1102.
2. (a) Niemeyer, C. M. Angew. Chem. Int. Ed. 2001, 40, 4128-4158. (b) Alivisatos, A. P. Nature Biotech. 2004, 22, 47-52. (c) Rosi, N. L.; Mirkin, C. A. Chem. Rev. 2005, 105, 1547-1562. (d) Medintz, I. G.; Uyeda, H. T.; Goldam, E. R.; Mattoussi, H. Nature Mater. 2005, 4, 435-446. (e) Michalet, X.; Pinaud, F. F.; Bentolila, L. A.; Tsay, J. M.; Doose, S.; Li, J. J.; Sundaresan, G.; Wu, A. M.; Gambhir, S. S.; Weiss, S. Science 2005, 307, 538-544.
3. (a) Ricco, A. J.; Crooks, R. M., Acc. Chem. Res. 1998, 31, 199-324. (b) Ellis, A. B.; Walt, D. R., Chem. Rev. 2000, 100, 2477-2738. (c) Fabbrizzi, L., Coord. Chem. Rev. 2000, 205, 1-232. (d) de Silva, A. P.; Tecilla, P., J. Mater. Chem. 2005, 15, 2617-2976.
4. Tomasulo, M.; Raymo, F. M. Org. Lett. 2005, 7, 4633-4636.
5. The diameter of the quantum dots was estimated from the wavelength (542 nm) of their band-gap absorption, according to a literature protocol (Yu, W.; Qu L.; Guo W.; Peng, X. Chem. Mater. 2003, 15, 2854-2860).
6. (a) Mangeney, C.; Ferrage, F.; Aujard, I.; Marchi-Artzner, V.; Jullien, L.; Ouari, O.; Rekaie, E. D.; Laschewsky, A.; Vikholm, I.; Sadowski, J. W. J. Am. Chem. Soc. 2002, 124, 5811-5821. (b) Abad, J. M.; Mertens, S. F. L.; Pita, M.; Fernandez, V. M.; Schiffrin, D. J. J. Am. Chem. Soc. 2005, 127, 5689-5694.
7. Uyeda, H. T.; Medintz, I. L.; Jaiswal, J. K.; Simon, S. M.; Mattoussi, H. J. Am. J. Chem. Soc. 2005, 127, 3870-3878.
8. The molar extinction coefficient of 3 is about 20.7 mM⁻¹ cm⁻¹ at 342 nm in CHCl₃. That of the quantum dots can be estimated to be about 97.5 mM⁻¹ cm⁻¹ at 542 nm in the same solvent, relying on a literature protocol (ref. 5).

Patents, patent applications, books, and other publications cited herein are incorporated by reference in their entirety. In particular, published after our priority date is Tomasulo et al. (J. Phys. Chem. B 110:3853-3855, 2006) and Tomasulo et al. (Langmuir 22:10284-10290, 2006).

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability.

All modifications and substitutions that come within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A composition of chemosensors for detection of an analyte, wherein at least one of said chemosensors comprises: (i) a fluorescent nanoparticle comprising an essentially spherical, semiconductor nanocrystal and (ii) one or more oxazine ligands in optical linkage such that fluorescence emission from the nanoparticle substantially overlaps with an absorption spectrum of at least one oxazine ligand or its phenolate derivative; wherein the analyte induces chemical transformation of the oxazine ligand to its phenolate derivative causing a shift in the absorption spectra between the oxazine ligand and the phenolate derivative.

2. The composition of claim 1, wherein at least one nanoparticle has a mean diameter from 1 nm to 10 nm.

3. The composition of claim 1, wherein the nanocrystal comprises CdSe or CdTe.

4. The composition of claim 1, wherein the nanoparticle is coated.

5. The composition of claim 4, wherein the nanoparticle is coated with an inorganic semiconductor layer and/or an organic layer.

6. The composition of claim 5, wherein the nanoparticle is coated with an inorganic semiconductor layer comprising ZnS or ZnSe.

7. The composition of claim 1, wherein at least one oxazine ligand has the formula: 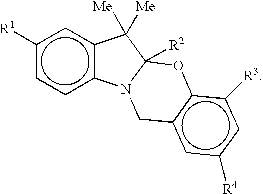
wherein R¹ appends the ligand to the nanoparticle, R² and/or R³ independently or coordinately bind the analyte, and R⁴ affects the absorption spectra of the ligand and/or its phenolate derivative.

8. The composition of claim 7, wherein R¹ is selected from the group consisting of amino, carbonyl, carboxyl, disulfide, hydroxyl, phosphate, and sulfhydryl linkers.

9. The composition of claim 7, wherein R² is selected from the group consisting of hydrogen, alkyls, cycloalkyls, substituted alkyls, substituted cycloalkyls, aryls, substituted aryls, heterocycles, substituted heterocycles, boronic acids, and polydentate chelators.

10. The composition of claim 7, wherein R³ is selected from the group consisting of hydrogen, alkyls, cycloalkyls, substituted alkyls, substituted cycloalkyls, aryls, substituted aryls, heterocycles, substituted heterocycles, boronic acids, and polydentate chelators.

11. The composition of claim 7, wherein R⁴ is a chromophore.

12. The composition of claim 1 further comprising a physiologically-acceptable carrier.

13. The composition of claim 12 which is apyrogenic and/or aseptic.

14. A kit, which is comprised of one or more containers in a package, further comprising (a) at least one composition of claim 1 and (b) a positive control or calibration standard of a known amount of analyte.

15. A method for detection of at least the presence or quantity of an analyte, said method comprising:
(a) contacting at least one chemosensor of claim 1 with a solution which might contain an analyte, wherein the analyte induces a chemical transformation of the oxazine ligand to its phenolate derivative;
(b) measuring whether or not there is a detectable change in fluorescent signal of the chemosensor; and
(c) correlating the change in fluorescent signal with detection of the analyte.

16. The method according to claim 15, wherein fluorescence of the nanoparticle is excited at one or more wavelengths from 300 nm to 700 nm.

17. The method according to claim 15, wherein fluorescent emission of the nanoparticle is measured at one or more wavelengths from 400 nm to 900 nm.

18. The method according to claim 15, wherein wavelength of maximum absorption by an oxazine ligand differs from wavelength of maximum absorption by its phenolate derivative by at least 150 nm.

19. The method according to claim 15, wherein fluorescence intensity and/or fluorescence lifetime is measured.

20. The method according to claim 15, wherein the chemosensor is contacted with analyte inside or outside of (i) an in vitro cultured cell or (ii) a cell or tissue in vivo.

21. A process of synthesizing of a chemosensor of claim 1.

22-23. (canceled)
